(19) United States
(12) Patent Application Publication
(10) Pub. No.: US 2022/0192809 A1
(43) Pub. Date: Jun. 23, 2022

(54) MOTILE CELL SORTING DEVICE

(71) Applicant: The University of Warwick, Coventry, West Midlands (GB)

(72) Inventors: Vasily KANTSLER, Coventry, West Midlands (GB); Max MEISSNER, Coventry, West Midlands (GB); Anton BUKATIN, Coventry, West Midlands (GB); Petr DENISSENKO, Coventry, West Midlands (GB)

(73) Assignee: The University of Warwick, Coventry, West Midlands (GB)

(21) Appl. No.: 17/604,249

(22) PCT Filed: Apr. 15, 2020
(86) PCT No.: PCT/GB2020/050955

§ 371 Date: Oct. 15, 2021

(30) Foreign Application Priority Data
Apr. 16, 2019 (GB) 1905373.5
Jan. 9, 2020 (GB) 2000314.1

(51) Int. Cl. A61D 19/02; C12M 1/00; C12M 1/12

(52) U.S. Cl. CPC A61D 19/022; C12M 21/06; C12M 23/04; C12M 23/34

(57) ABSTRACT

A motile cell sorting device is disclosed. The device comprises a chamber, an inlet and an outlet in fluid communication with the chamber, and a plurality of discrete barriers disposed in the chamber. Each discrete barrier comprises at least one wall and at least one acute edge orientated towards the outlet.

### FIELD

The present invention relates to a motile cell sorting device.

### BACKGROUND

Increasingly, couples are tending to wait until they are older before they begin trying to start a family. The longer a couple wait, however, the lower the chances of successfully conceiving. Thus, assisted reproduction treatments are becoming ever-more important (both from personal view and on a wider, societal level) since they can help increase the chances of conceiving.

Assisted reproduction treatments generally fall into two categories.

Intrauterine insemination (IUI) is a process whereby a prepared sperm sample is introduced into the (female) uterus using a catheter and fertilisation takes place in the uterus. This approach can be used to treat both male-factor and female-factor fertility problems. Although it is generally less invasive than other comparable treatments, it is being used less often due to lower success rates.

In vitro fertilisation (IVF) is a process involving combining a prepared sperm sample with an oocyte (egg) to create embryos in a laboratory setting. IVF can be divided further into two distinct treatment procedures, namely conventional IVF involving combining oocytes with a prepared sperm sample (typically 50,000 to 100,000 sperm cells) in a laboratory dish, where fertilisation takes place and IVF with intracytoplasmic sperm injection (ICSI) involving selecting a single sperm cell from a prepared sperm sample and injecting it directly into the oocyte. If successful, the outcome of either IVF procedure is a fertilised egg which is allowed to develop into an embryo for three to five days in a special culture medium in a controlled environment, before being transferred to the uterus for potential implantation and embryo development.

Assisted reproduction generally employ a sperm preparation or “sperm washing” step. The objectives of this step include isolating the sperm cells from the seminal fluid, which can contain undesirable contaminants (including cellular debris, bacteria, immune cells, mucus and other chemicals which could adversely affect the chance of successful fertilisation), removing any cryopreservative chemicals (if the sperm sample has been frozen) and selecting only motile sperm cells and preferably the most motile sperm cells from a sample.

Generally, there are three ways of performing sperm separation.

Simple washing involves a suspending sperm in an appropriate sperm-washing medium, then performing centrifugation to collect the sperm cells. Although this approach successfully dilutes chemical contaminants, it tends not to remove dead cells or cellular debris and does not separate out living cells from dead cells.

In density gradient centrifugation (DGC), samples are centrifuged in a test tube containing fluids of varying density. The fluids are calibrated in such a way that only cells of the correct density are collected, and cellular debris or heavily damaged cells are left behind. Intact and swimming cells normally have a slightly higher density and so this approach can be used to separate cells based on the density difference, but not directly on motility characteristics.

In so-called “swim-up”, a sample of appropriate sperm washing medium is carefully floated on top of a semen sample which has been gently pelleted by centrifugation. The motile cells swim up into the washing medium and the non-motile cells remain in the pellet.

The current washing methods tend to suffer one or more problems.

First, they tend to involve at least one centrifugation step, which is thought to cause DNA damage to the cells. Secondly, swim-up is not selective for progressive motility and so can result in lower quality of the selected spermatozoa. Thirdly, while DGC can separate out motile cells with some degree of specificity, the efficiency of the process is variable and depends on a number of factors including, for example, how many different fluids are used, the densities of those fluids, the centrifuge speed, and the skill of the technician. It has also been shown that DGC can increases DNA damage, which can further affect embryo survival rate.

A number of alternatives to the washing methods are emerging.

One approach is to use microfluidic separation. Examples of sperm separation devices include the ZyMōt™ Multi and ZyMōt™ ICSI available from ZyMōt Fertility (Gaithersburg, Md. USA). These devices function either by using wall reflection of sperm cells along a narrow channel or by using a thin membrane to assist swim up.

Another approach involves rheotactic separation involving exposing cells to a gentle fluid flow along which sperm cells orient themselves resulting in them swimming into a collection chamber.

Yet another approach, electrophoresis, uses an electric field to separate out cells based on their dielectric constant. Magnetic separation can also be used, although this approach relies primarily on binding magnetic particles to specific cells and subsequently separating the cells.

A method of separating motile sperm is described in WO 2016/035799 A1.

Reference is also made to WO 2017/127775 A1.

### SUMMARY

According to a first aspect of the present invention there is provided a motile cell sorting device. Motile cells may be spermatozoa and may be human, equine, bovine, porcine or avian. The device comprises a chamber, an inlet and an outlet in fluid communication with the chamber, and a plurality of discrete barriers disposed in the chamber. Each discrete barrier comprises at least one wall and at least one acute edge orientated towards the outlet.

This can allow not only the most motile cells, but also the progressively motile cells to be isolated and concentrated while minimising or even avoiding chemical, electrical, thermal and/or gravitational gradients and so help to reduce the risk of cell damage; these types of cells tend to be closely correlated with pregnancy success rates and lower miscarriage rates. This can also help with preferentially separating out cells deemed to have acceptable morphology and structure.

The barriers are (in plan view) preferably crescent-shaped or arrowhead-shaped. However, the barriers may be teardrop-shaped, semi-circular or chevron-shaped.

The at least one wall may comprise first and second walls and the at least one acute edge may comprise a first acute edge between the first and second walls. The first wall may be convex, straight or concave. The second wall may be concave or straight. The at least one acute edge may further comprise a second acute edge. The second acute edge may between the first wall and the second wall, for example, forming a crescent-shaped barrier. The at least one wall may comprise a third wall and the second acute edge may be between the second and third walls.

An acute edge is defined by two walls (or two portions of a wall) meeting at an angle of greater than 0° and less than 90°. Preferably, the angle is less than 30°. The curvature of each acute edge may be greater than 0 μm and less than or equal to 50 μm 0 and preferably less than 20 μM.

The diameter of the device may be between 2.5 cm and 3.5 cm.

The channel 4 of the device may be less than 100 μm.

The chamber may comprise a channel running between the inlet and the outlet provided at first and second ends respectively and comprising first and second chamber walls. The chamber may be disk-shaped having a periphery and a centre and wherein the inlet is annular and arranged around the periphery of the chamber and the outlet is arranged at the centre. The chamber preferably has a height which is between 50 to 300 μm. Preferably, the chamber height is greater than 100 μm.

The inlet and/or outlet may be between 1 and 8 mm in diameter. The inlet may be 3 mm in diameter. The inlet may be 5 mm in diameter. The outlet may be 5 mm in diameter.

Each discrete barrier preferably has a width of between 10 and 500 μm, or between 100 and 150 μm. Each discrete barrier may have a width of 125 μm. Each discrete barrier preferably has a length of between 10 and 1000 μor between 100 and 250 μm. Each discrete barrier may have a length of 175 μm. Each discrete barrier preferably is separated from neighbours in a first direction (e.g., in a row) by a first gap of between 20 to 500 μm, and or between 100 to 500 μm. Each discrete barrier preferably is separated from neighbours in a second different direction (e.g., in a line or column) by a second gap of between 20 to 500 μm, or between 100 to 500 μm. The discrete barriers may project into the chamber from a floor or a ceiling. The discrete barriers may be identically-shaped. The discrete barriers may be arranged in a periodic array, which may be rectangular or hexagonal array.

According to a second aspect of the present invention there is provided an intrauterine insemination kit comprising the device of the first aspect.

According to a third aspect of the present invention there is provided a method of using the device of the first aspect, the method comprising supplying a sample comprising motile cells to the inlet, waiting for a period of time of at least 1 minute and, after waiting for the period of time, collecting a refined sample from the outlet.

The period of time may be at least 5 minutes and is preferably at least 10 minutes. The period of time may be between 10 to 200 minutes or 10 to 120 minutes or between 10 and 60 minutes. The method may further comprise causing the device to be heated to a temperature for incubation. The temperature for incubation may be 37° C.

The sample and/or device may be purged and/or washed with one or more buffers.

The sample may be washed with buffers to reduce the probability of the spermatozoa cells sticking to each other.

### BRIEF DESCRIPTION OF THE DRAWINGS

Certain embodiments of the present invention will now be described, by way of example, with reference to the accompanying drawings, in which:

FIG. 1 is a perspective view of a microfluidic chip having first and second ports and a microchannel running between the first and second posts and containing entraining structures;

FIG. 2 is a perspective view of an array of entraining structures;

FIG. 3 is a plan view of a first type of entraining structure;

FIG. 4 is a second type of entraining structure;

FIG. 5 is a plan view of a microchannel containing an array of the first type of entraining structure;

FIG. 6 is a plan view of a microchannel containing an array of the second type of entraining structure;

FIGS. 7a and 7b are plan views of further examples of entraining structure;

FIG. 8 is a plan view of yet another example of an entraining structure;

FIGS. 9a to 9c is a plan view of wall structures;

FIG. 10a are photographs of a first device and the entraining structures;

FIG. 10b are photographs of a fisecondrst device and the entraining structures;

FIG. 11 is a process flow diagram of a method of sorting and extracting spermatozoa;

FIG. 12 is a process flow diagram of a method of sorting and extracting spermatozoa;

FIG. 13 is a table of results of sample analysis;

FIG. 14 is a table of results of sample analysis;

FIG. 15 is a table of results of sample analysis;

FIG. 16 is a table of results of sample analysis;

FIG. 17 is a table of summary results of sample analysis;

FIG. 18 is a table of summary results of sample analysis;

FIG. 19 is a bar chart of spermatozoa motility;

FIG. 20 is a bar chart of spermatozoa motility;

FIG. 21 is a bar chart of spermatozoa morphology;

FIG. 22 is a bar chart of spermatozoa morphology;

FIG. 23 is a bar chart of spermatozoa DNA fragmentation;

FIG. 24 is a bar chart of spermatozoa DNA fragmentation;

FIG. 25 are photographs of spermatozoa DNA fragmentation;

FIG. 26 are photographs of spermatozoa DNA fragmentation.

### DETAILED DESCRIPTION OF CERTAIN EMBODIMENTS

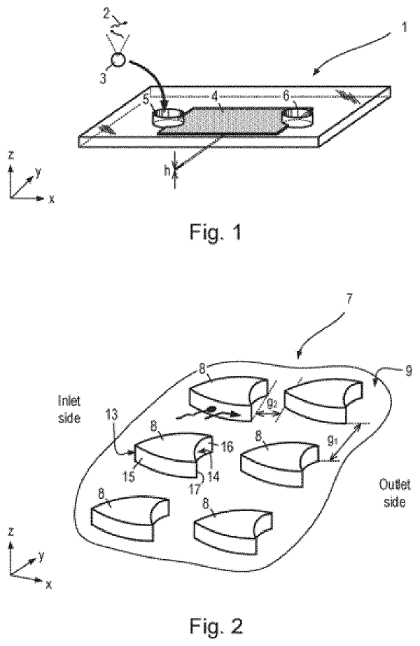

Referring to FIG. 1, a device 1 for sorting motile cells 2, such as spermatozoa, in a sample 3 is shown.

The device 1 includes a chamber 4, for example in the form of a low-height channel or disc, an inlet 5, and an outlet 6 in fluid communication with the channel 4. The chamber 4 has a height which is less than, preferably much less than (at least by a factor 10 or even 100) its lateral dimensions, such a length and width. The chamber 4 has a height, h, which is preferably between 50 to 300 μm. The chamber 4, inlet 5 and outlet 6 are arranged such that when a sample 3 containing motile cells 2 is supplied to the inlet 5, motile cells 2 swim through the chamber 4 towards the outlet 6. As they swim through the chamber 4, the motile cells 2 are sorted and separated on the basis of motility such that less motile cells 2 (e.g., immotile cells) tend to be retained in the chamber 4, while more motile cells 2 tend to progress along the chamber 4.

Referring also to FIG. 2, the device 1 includes an arrangement 7 of discrete barriers 8 (or “ratchet”) disposed in the chamber 4 projecting into the chamber 4 from a first inner surface 9, e.g., the floor of the chamber 4, to a second, opposite inner surface, e.g., its ceiling. The arrangement 7 preferably takes the form of a periodic two-dimensional array, such as a hexagonal or cubic lattice. The barriers 8 are separated from side neighbours by a first gap, g₁, of between 10 to 500 μm and from neighbours in front and behind by a second gap, g₂, of between 10 to 500 μm

Each barrier 8 is generally asymmetrical having differently-shaped first and second faces 13, 14 orientated towards and away from the first port 5 respectively. The first face 13 includes at least one wall 15 and the second face 14 preferably includes at least one wall 16. Herein, the walls 15, 16 may be referred to as “side walls”. The walls 15, 16 or opposite ends of the wall 15 meet at one or more acute edges 17 (herein referred to as “discontinuities”). The angle between the walls 15, 16 is greater than 0° and less than 90° and is preferably less than 30°. The curvature of the edge 17 is greater than 0 μm and less than or equal to 50 μm, micron and is preferably less than 20 μm.

The chamber 4, inlet 5, outlet 6 and barriers 8 are configured such that motile cells 2 take a time, t, of between 10 to 60 minutes, preferably about 20 minutes, to swim from the inlet 5 through the chamber 4.

The device 1 may operate at ambient temperature, i.e., room temperature. However, the device 1 may be provided with a heater (not shown), for example in the form of hot plate, oven or water bath, to elevate the operating temperature of the device to a suitable temperature for incubation, for example, about 37° C.

Referring in particular to FIG. 1, the device 1 preferably takes the form of a microfluidic chip. The device 1 may comprise an assembly of first and second planar portions (not shown) formed from glass and/or or polymeric materials, such as polydimethylsiloxane (PDMS), polymethyl methacrylate (PMMA) or poly octanediol-co-citrate (POC). The first portion (which may be referred to as a “base”) may have a patterned face (not shown) defining a bottom (or “floor”) and sides of the chamber 4 and the barriers 8. The second portion (which may be referred to as a “cover”) may be featureless (e.g., flat) and may define a top (or “ceiling”) of the chamber 4. The cover may also include first and second ports (not shown) an unrefined sample and a refined sample may be provided and collected respectively. The first and second portions (not shown) may consist of the same material or different materials. The device 1 may be fabricating in different ways, for example, by moulding or 3D-printing.

The barriers 8 utilize surface entrainment, whereby motile cells 2 tend to swim along a surface, to sort the motile cells. The barriers 8 have curved surfaces 15, 16 with sharp discontinuities to redirect swimming cells along the desired movement orientation.

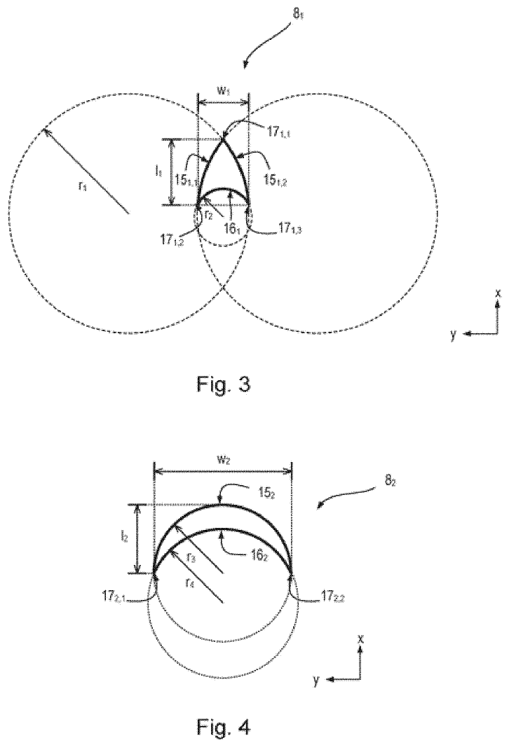

Referring to FIGS. 3 and 4, first and second barrier shapes 8₁, 8₂ are shown

Referring in particular to FIG. 3, the first shape 6₁ generally has an arrowhead-like geometry having two convex segments 15_{1,1}, 15_{1,2} joined at a first edge 17_{1,1} and one concave segment 16₁ joined to the convex segments 9_{1,1}, 9_{1,2} via sharp, second and third edges 17_{1,2} 17_{1,3}. To sort cells, the barrier 8₁ is oriented such that the side with a single edge 17_{1,1} points towards the inlet 5 of the device 8₁. Cells swimming in the desired direction are gently guided along the barrier 8₁ while cells going the wrong direction are turned around by the concave segment 16₁ and reoriented towards the outlet 6.

The first barrier 8₁ is defined by half the intersection of first and second intersecting virtual circles 18₁, 18₂ having a cut out defined by a third circle 18₃. The convex walls 15_{1,1}, 15_{1,2} are defined by intercepted arcs of the first and second overlapping virtual circles 18₁, 18₂ each having a first radius r₁. The concave wall 16₁ is defined by the intercepted arc of a third virtual circle 18₃ having a second radius r₂. In this case, r₂<r₁. The first barrier 8₁, has a width w₁ of between 10 and 500 μm and a length l₁ of between 10 and 1,000 μm. In this example, r₁=450 μm, r₂=82 μm, w₁=150 μm and l₁=177 μm.

Referring in particular to FIG. 4, the second shape 8₂ generally has a crescent moon-like geometry having one convex segment 15₂ and one concave segment 16₂ joined via sharp edges 17_{2,1}, 17_{2,2}. The edges 17_{2,1}, 17_{2,2} are orientated orient towards the outlet 6 of the device. Cells which encounter the convex side are simply guided along, while cells which encounter the concave side are re-oriented towards the outlet.

The second barrier 8₂ is defined by third and fourth overlapping virtual circles 18₃, 18₄. The convex and concave walls 15₂, 16₂ are defined by fourth and fifth arcs of the third and fourth circles 18₃, 18₄ having a third radius r₃ and a fourth radius r₄ respectively. The first barrier 8₁, has a width w₂ of between 10 and 500 μm and a length l₂ of between 10 and 1,000 μm. In this example, r₃=125 μm, r₄=137 μm, w₂=250 μm and the two circles are offset by 39 μm.

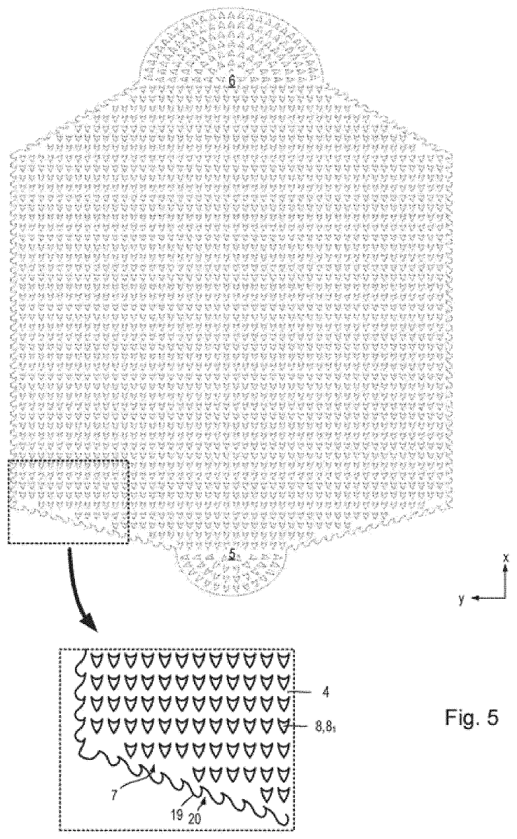

Referring to FIG. 5, the chamber 4 takes the form of a channel which is generally linear, running along a path (which may be straight, curved or include bends) between the inlet 5 and outlet 6. The channel 4 has a wall 19 having repeated barbs 20, in this case having a shark-fin-like shape. A rectangular array 7 of barriers 8 of the first shape 8₁ is used. A different type of array, e.g. hexagonal, and/or a different barrier shape, e.g. the second shape 8₂, can be used. The channel 4 may have a different shape.

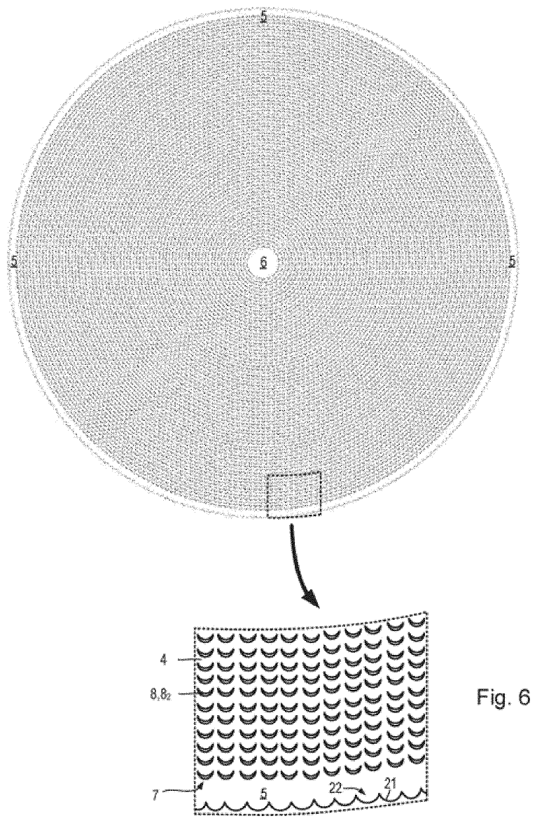

Referring to FIG. 6, the chamber 4 may be radial (or “circular”) whereby the motile cells are introduced around a peripheral, annular inlet 5 and swim inwardly towards a central outlet 6. The chamber 4 is defined by a generally circular wall 21 having scallops 22. A radial array 7 of barriers 8 of the second shape 8₂ is used. A different type of array, e.g. hexagonal, and/or a different barrier shape, e.g. the first shape 8₁, can be used.

Referring again to FIG. 3, the first shape 6₁ of barrier generally has an arrowhead-like geometry having a first face 13 which comprises two convex walls 15_{1,1}, 15_{1,2}. However, these walls can have other, different shapes.

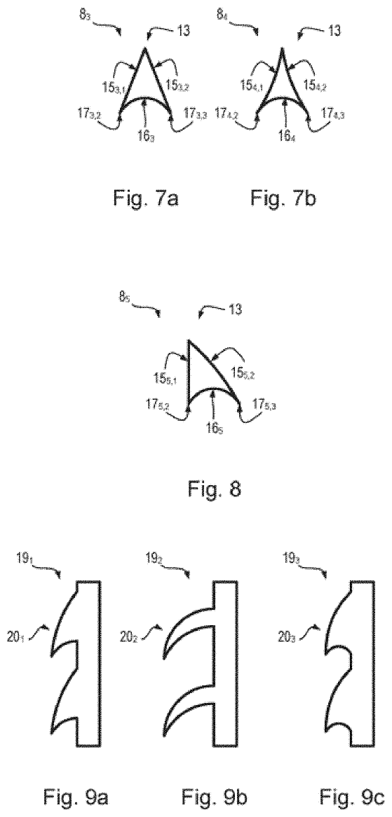

Referring FIGS. 7a to 7b, the first face 13 may have straight walls 15_{3,1}, 15_{3,2} or even concave wall 15_{4,1}, 15_{3,2} meeting the concave wall 16₃, 16₄ at acute edges 17_{3,2}, 17_{3,3}, 17_{4,2}, 17_{4,3}. For example, the barb may be a half arrowhead, half crescent or half a modified arrowhead (the full modified arrowhead having two concave walls)

Referring to FIG. 8, if there are two or more walls in the first face 13, the walls 15_{5,1}, 15_{5,2} need not have the same shape. For example, one wall 15_{5,1} may be straight and the other wall 15_{5,2} may be convex. Thus, the edges 17_{5,2,} 17_{5,3} may have different sharpnesses.

Referring to FIGS. 9a to 9c, the walls 19₁, 19₂, 19₃ of the chamber 4, can have different shapes of barbs 20₁, 20₂, 20₃ which may be based on the shapes of the barriers herein described.

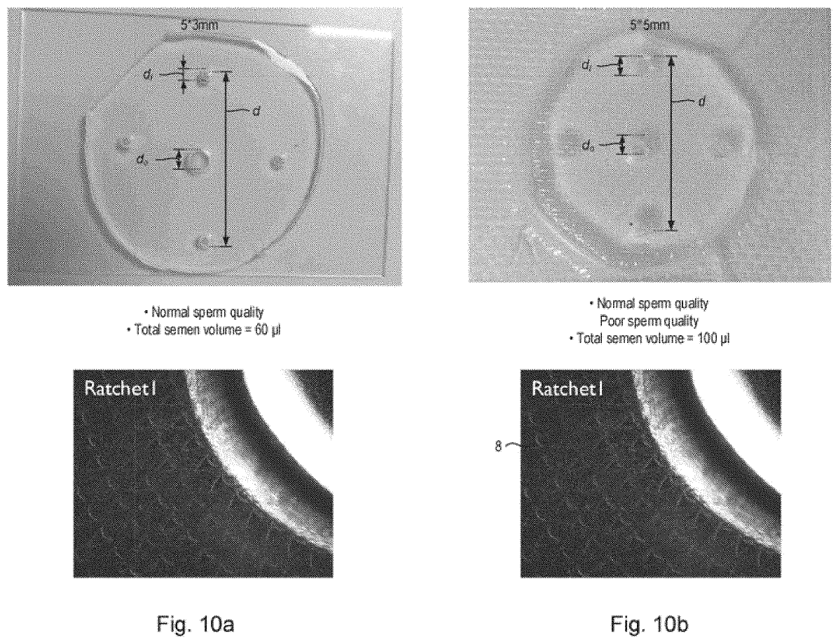

Referring to FIGS. 10a and 10b, photographs of a device 1 similar to that shown in FIG. 6 and photographs of barriers 8₁ as shown in FIG. 3 used in these devices are shown. FIG. 10a illustrates a device 1 having inlets 5 with a diameter dᵢ of 3 mm, and an outlet 6 having a diameter d₀ of 5 mm. This device 1 is also referred to as a 5*3, or 5*3 mm device. The diameter d of the device 1 is between 2.5 cm and 3.5 cm. The 5*3 device 1 includes barriers 8₁, as illustrated in FIG. 3, spaced at a pitch of greater than 100 μm, however, the barriers may have a larger or smaller pitch depending on the sample used. A higher magnification photograph of the barriers 8₁ in the device 1 is shown in the lower part of FIGS. 10a and 10b. A device with these arrowhead-like barriers 8₁ may also be referred to as “Ratchet 1”, or more simply “R₁”. The channel 4 of the 3*5 device 1 is less than 100 μm. FIG. 10b illustrates a device 1 having inlets 5 with a diameter dᵢ of 5 mm, and an outlet 6 having a diameter d₀ also of 5 mm. This device 1 is also referred to as a 5*5, or 5*5 mm device. The diameter d of the device 1 is 3.5 cm. The barrier 8 structures, pitch and channel 4 are the same in the 3*5 device and the 5*5 device.

The 5*3 device can yield better results as fewer undesirable cells and other debris are removed at the extraction stage, however, it can be more difficult to extract a sample from the 5*3 device than from the 5*5 device. The 5*5 device can yield a lower quality sample than the 5*5 device, but the sample can be easier to extract from the device.

The inlet 5 diameter dᵢ and the outlet diameter d₀ may be between 1 and 8 mm. The diameter d of the device may be between 1 and 10 cm. The sizes of the inlets and outlets can be optimized for the quality and/or type of each sample.

Referring also to FIG. 3, the barrier 8₁ in the devices in FIGS. 10a and and 10b may have a width w₁ of 125 μm and length lᵢ of 175 μm.

The 3*5 device 1 was used to isolate high quality spermatozoa in semen with normal quality spermatozoa using semen samples of 60 μl, and the 5*5 device was used to isolate high quality spermatozoa in semen with normal and poor quality spermatozoa using semen samples of 100 μl.

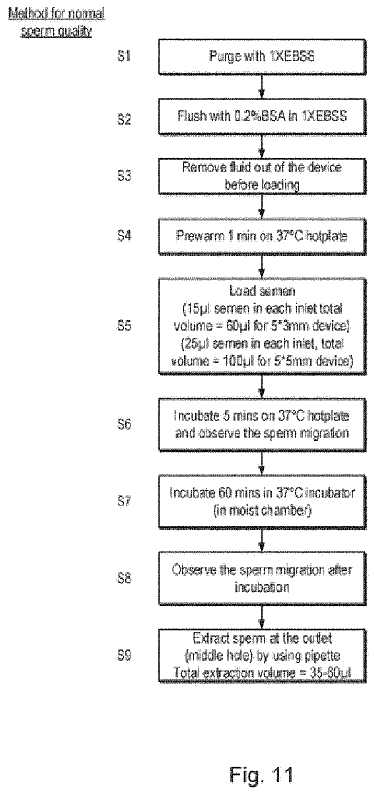

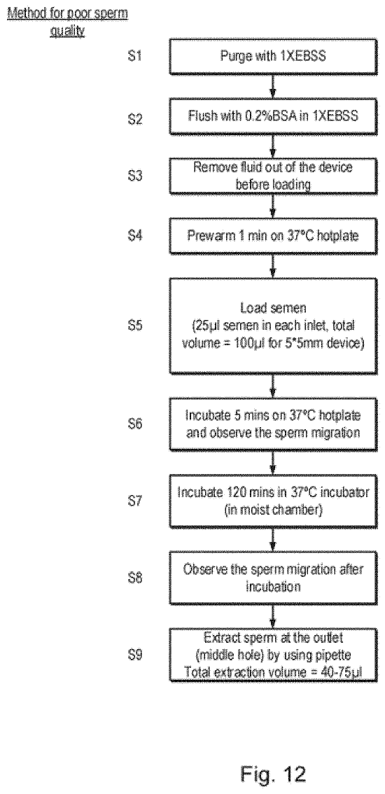

Referring to FIGS. 11 and 12, normal and poor quality sperm semen samples are collected and inserted into the device 1 to isolate higher quality sperm.

Sperm were recovered from 14 ejaculates. Ejaculates were divided into normal and abnormal groups based on sperm progressive motility (PR) by World Health Organisation criteria. Sperm concentration, progressive motility, total motility, morphology and DNA fragmentation (using Terminal deoxynucleotidyl transferase TdT dUTP Nick End Labeling (TUNEL) assay) were measured in the sperm selected by all methods and in the semen sample.

Referring specifically to FIG. 11, steps are illustrated for the method of isolating high quality spermatozoa in normal sperm quality samples using the device 1. In step S1, the sample is purged using a buffer, for example, Earle's Balanced Salt solution (1×EBSS). Other suitable buffers may be used and the type and concentration will depend on the sample and desired outcome of the process. In step S2, the sample is flushed with a buffer to reduce the change of spermatozoa sticking to each other, for example, a 0.2% bovine serum albumin (BSA) in 1×EBSS. However, other suitable buffers which have the effect of reducing the probability of spermatozoa sticking to each other may be used. In step S3, if there is excess fluid in the sample at this stage, this fluid may be removed from the sample before loading into the device. In step S4, the sample and/or the device is warmed on a 37° C. hotplate. In step S5, the semen sample is divided into four equal parts and loaded into the four inlets 5 of the device 1, however, it may be that only some inlets 5 have a portion of the sample loaded into them. In step S6, the device 1 containing the sample is incubated for 5 minutes at 37° C. and sperm migration is observed either manually using a microscope or automatically using a microscope and video camera to confirm the sample has been loaded correctly. Other methods of confirming that the sample has been loaded correctly may be used, for example, via an optical or movement sensor at the beginning of step S7. In step S7, the device containing the sample is incubated for 60 minutes. In step S8, the sperm is observed either manually or automatically after incubation. In step S9, the sperm is extracted from the device 1 through the outlet 6 using a pipette. The volume extracted from the outlet 6 may be between 35 and 60 μl.

Referring specifically to FIG. 12, steps are illustrated for the method of isolating high quality spermatozoa in poor sperm quality samples using the device 1. For the poor quality sperm samples, steps S1 to S6 are the same as for the normal quality sperm samples. In step S7, the device containing the sample is incubated for 120 minutes. Steps S8 and S9 are the same as for the normal quality sperm samples. The volume extracted from the outlet 6 may be between 40 and 75 μl.

Alternative processes may be used in a working device. The steps in the processes outlined above may be modified or removed, for example, step S3, may be removed altogether. Additional steps may also be added, for example, additional washing or purging of a sample and/or device with additional buffers. The timings, temperatures and volumes of sample associated with steps in the processes may be optimized for particular samples, species, or aim of the process, for example the incubation time may be longer or shorter in step S7, depending on the sample type, size, and speed of sperm. The volumes extracted from the device at the end of the process may be higher or lower depending on the types and volumes of samples used and the types and volumes of buffers added to the sample. In a high throughput system, step S6 may also be removed, or combined with step S8, where the observation of the sample is performed at the beginning of step S8.

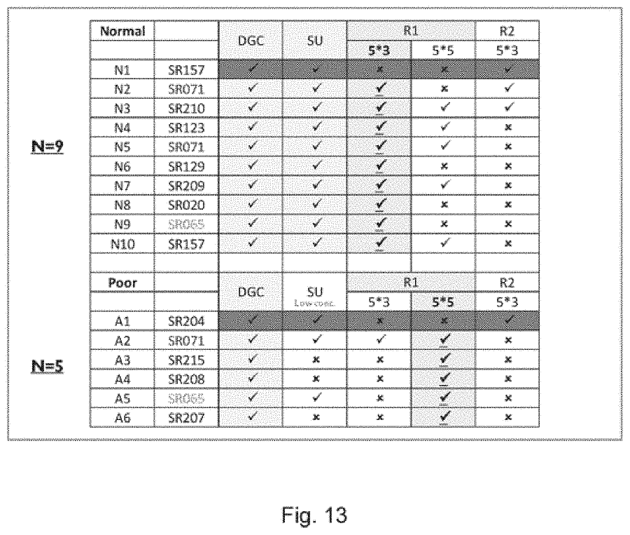

Referring to FIG. 13, a table indicates whether a process of a semen sample was successful (indicated by a tick) or not (indicated by a cross). Samples from ten individuals (N1 to N10) with normal quality sperm and six individuals (A1 to A6) with poor quality sperm underwent five methods for isolating high quality sperm. These methods were density gradient centrifugation (DGC), swim up (SU), a method using a 5*3 device and a 5*5 device both with barriers 8₁ as illustrated in FIGS. 2 and 3 (also labelled “R1”), and a method using a device with barriers 8₂ illustrated in FIG. 4 (labelled “R2”). As can be seen from the table, nine samples of normal quality sperm were successfully processed using the 5*3 device 1 and five samples of poor quality sperm were successfully processed using the 5*5 device 1. A total of 4 samples form both normal and poor quality groups were successfully processed using the R2 method.

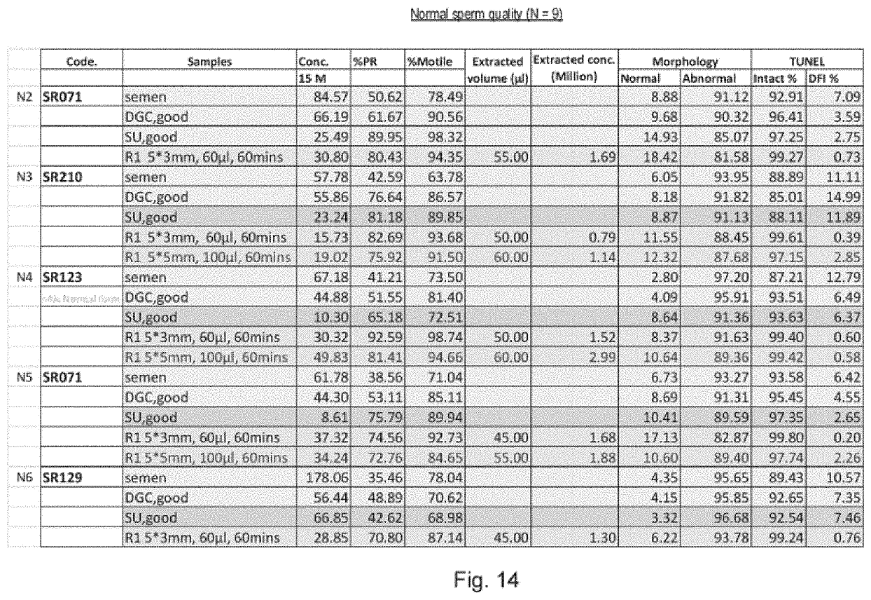

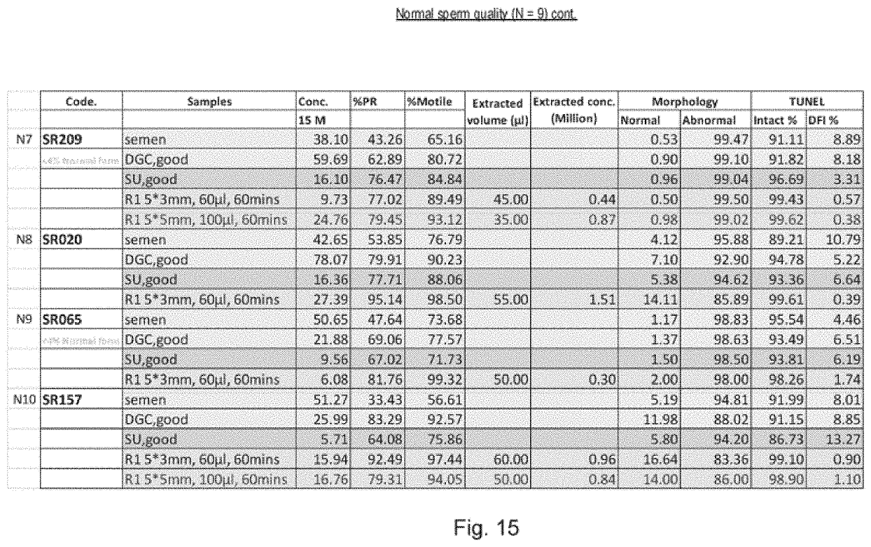

Referring to FIGS. 14 to 16, the results from analyses of the samples are presented in tables. The results show the concentration, the percentage progressive motility (% PR), the percentage motility (% Motile), extracted volume, the extracted concentration, percent of sperm showing normal and abnormal morphology (based on World Health Organisation classifications), and the percent of sperm intact or fragmented based on the TUNEL method.

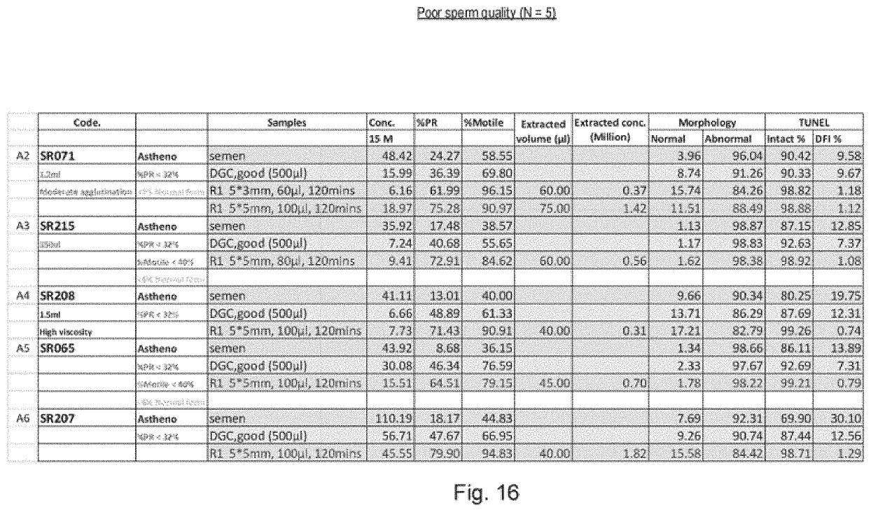

Referring in particular to FIG. 16, the table detailing the results for the poor quality sperm also indicates whether the sperm has a low motility condition classified as Astheno.

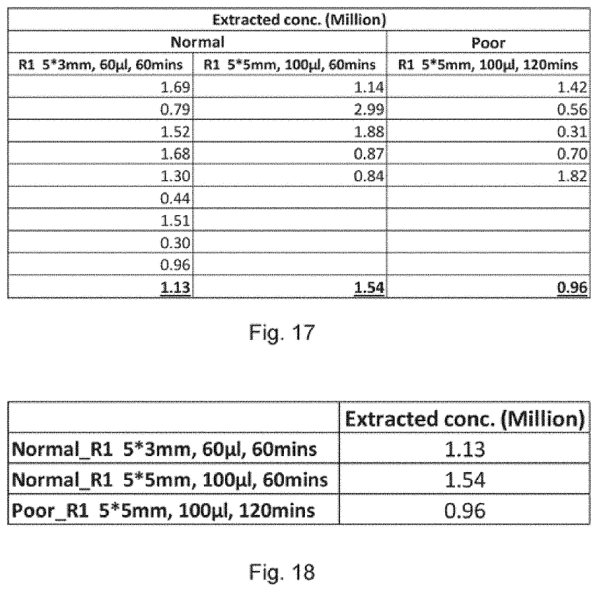

Referring to FIGS. 17 and 18, the results of the methodologies presented in FIGS. 13 to 16 are presented in summary form.

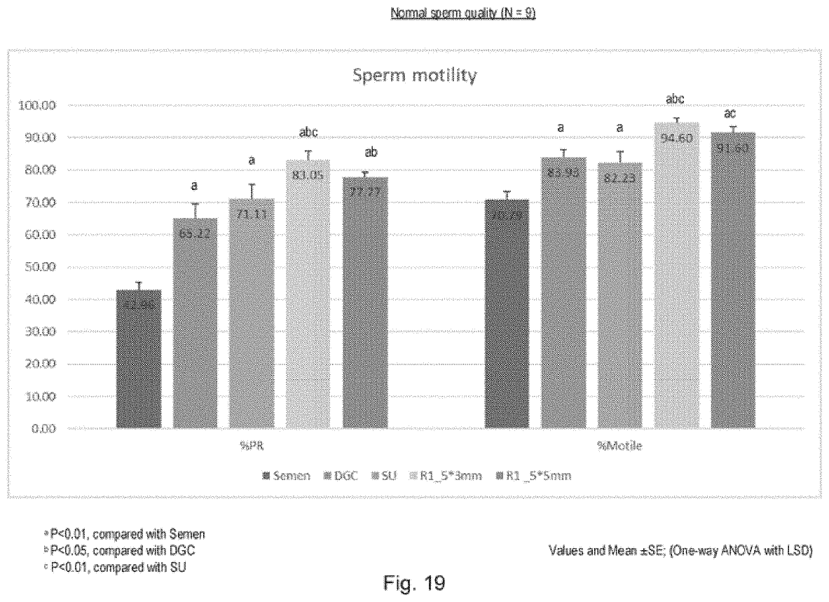

Referring to FIG. 19, a bar chart illustrates the percentage progressive motility and percentage motility±the standard error of the mean (±SEM) for the results for normal quality sperm from the methods in the table shown in FIG. 13 and also unprocessed semen. The Figure also contains the results from statistical analysis showing that using a One-way ANOVA with LSD (one-way analysis of variance with least significant difference), the R1 5*3 device yields a sample with significantly higher motility and progressive motility than a raw sample, a sample having undergone density gradient centrifugation and a sample having undergone the swim up method.

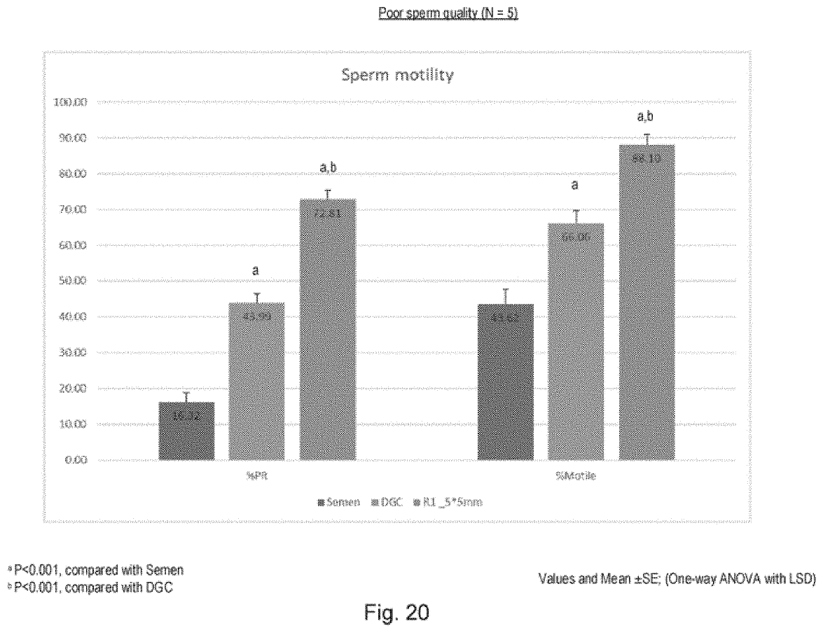

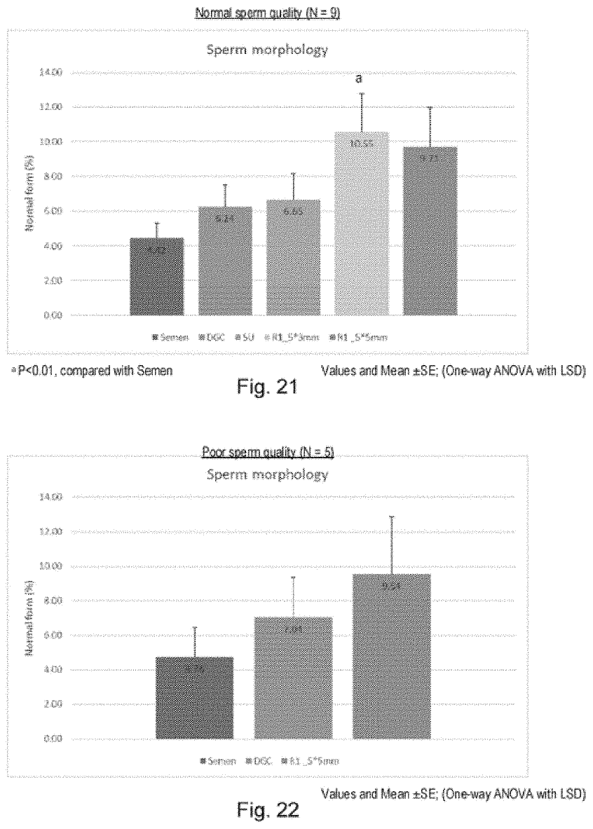

Referring to FIG. 20, a bar chart illustrates the percentage progressive motility and percentage motility±the standard error of the mean (±SEM) for the results for poor quality sperm from three of the methods in the table shown in FIG. 13 and unprocessed semen, specifically density gradient centrifugation and the method using the R1 5*5 device. The One-way ANOVA with LSD also shows a significant difference in percentage progressive motility and motility between the sample extracted from the R1 5*5 device and both the unprocessed semen and the semen having undergone density gradient centrifugation.

Referring to FIG. 21, a bar chart illustrates the percentage normal morphology±the standard error of the mean for the results for normal quality sperm from the methods in the table shown in FIG. 13 and also unprocessed semen. A One-way ANOVA with LSD also shows a significant difference in percentage normal morphology of those samples extracted from the R5*3 device and unprocessed semen.

Referring to FIG. 22, a bar chart illustrates the percentage normal morphology±the standard error of the mean for the results for poor quality sperm having undergone density gradient centrifugation and processing using the R1 5*5 device and also unprocessed semen.

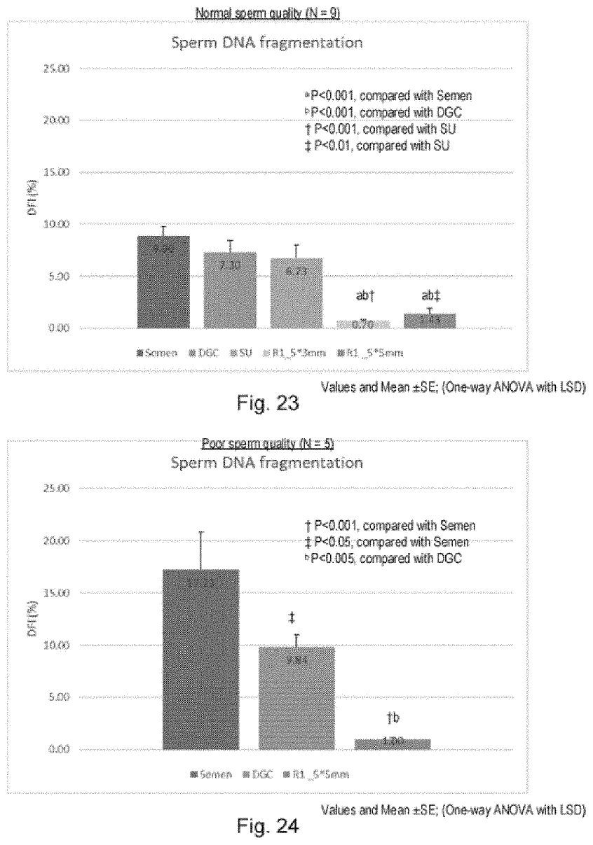

Referring to FIG. 23, a bar chart illustrates the percentage sperm fragmentation±the standard error of the mean for the results for normal quality sperm from the methods in the table shown in FIG. 13 and also unprocessed semen. A One-way ANOVA with LSD also shows a significant difference in percentage fragmentation of those samples extracted from the R1 5*3 and R1 5*5 devices compared to the other methods and unprocessed semen.

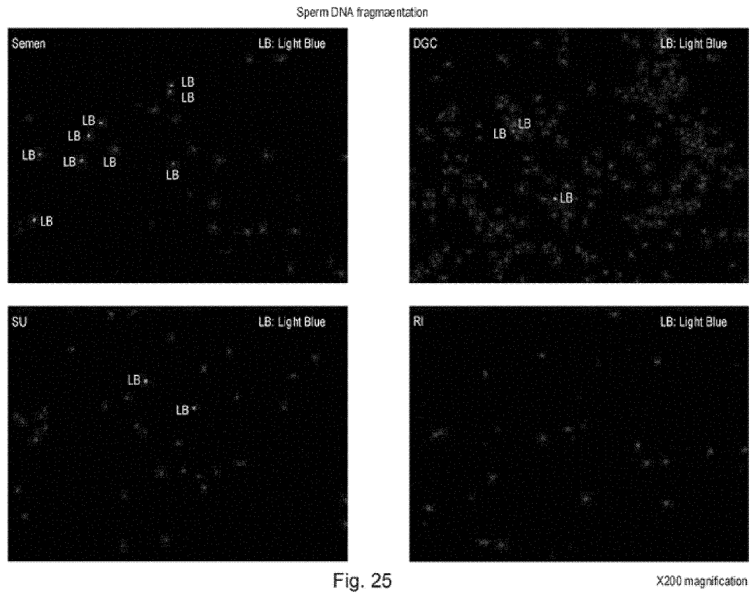

Referring to FIG. 24, a bar chart illustrates the percentage sperm fragmentation±the standard error of the mean for the results for poor quality sperm having undergone density gradient centrifugation and processing using the R1 5*5 device and also unprocessed semen. A One-way ANOVA with LSD also shows a significant difference in percentage fragmentation of those samples extracted from the R1 5*5 device compared to samples having undergone density gradient centrifugation and unprocessed semen.

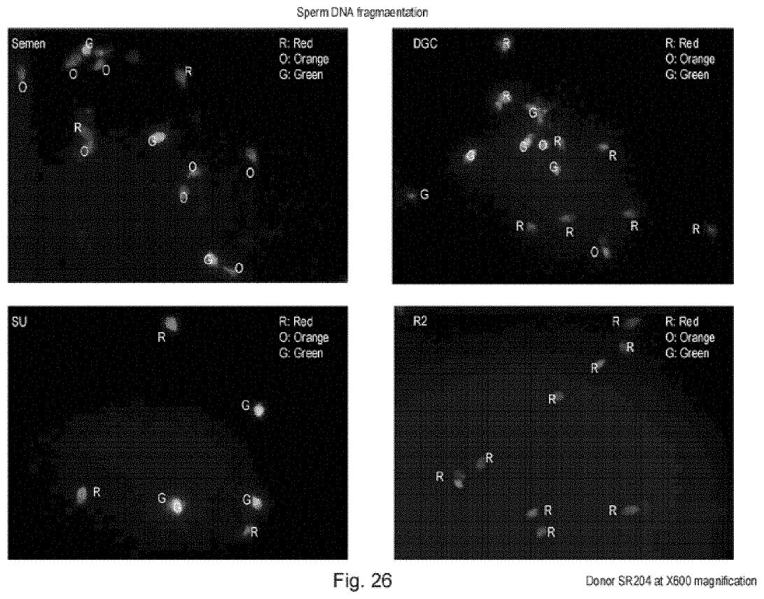

Referring to FIG. 25, photographs of fragmented semen at 200× magnification are shown for unprocessed semen, and samples having undergone density gradient centrifugation, swim up and processing using the R1 5*3 device. Fragments are stained to appear light blue and labelled “LB”.

Referring to FIG. 26, photographs of fragmented semen at 600× magnification are shown for unprocessed semen, and samples having undergone density gradient centrifugation, swim up and processing using the R2 5*3 device. Fragments are stained green (indicated here with a “G”), partially fragmented sperm are stained orange (indicated here with an “O”) and unfragmented and undamaged sperm are stained red, (indicated here with an “R”).

Modifications

It will be appreciated that various modifications may be made to the embodiments hereinbefore described. Such modifications may involve equivalent and other features which are already known in the design, manufacture and use of motile cell sorting devices and component parts thereof and which may be used instead of or in addition to features already described herein. Features of one embodiment may be replaced or supplemented by features of another embodiment.

Concave or convex surfaces need not be defined by arcs of circle. For example, a surface may be defined by an arc of an ellipse, a hyperbola or other suitable curve. The curvature may vary along the surface.

Although claims have been formulated in this application to particular combinations of features, it should be understood that the scope of the disclosure of the present invention also includes any novel features or any novel combination of features disclosed herein either explicitly or implicitly or any generalization thereof, whether or not it relates to the same invention as presently claimed in any claim and whether or not it mitigates any or all of the same technical problems as does the present invention. The applicants hereby give notice that new claims may be formulated to such features and/or combinations of such features during the prosecution of the present application or of any further application derived therefrom.

1. A motile cell sorting device comprising:
a chamber;
an inlet and an outlet in fluid communication with the chamber; and
a plurality of discrete barriers disposed in the chamber, wherein each discrete barrier comprises at least one wall and at least one acute edge orientated towards the outlet.

2. The device of claim 1, wherein the at least one wall comprises:
first and second walls; and
wherein the at least one acute edge comprises:
a first acute edge between the first and second walls.

3. The device of claim 2, wherein the first wall is convex, straight or concave.

4. The device of claim 2, wherein the second wall is concave.

5. The device of claim 2, wherein the second wall is straight.

6. The device of claim 2, wherein the at least one acute edge further comprises:
a second acute edge.

7. The device of claim 6, wherein the second acute edge is between the first wall and the second wall.

8. The device of claim 7, wherein the at least one wall comprises:
a third wall;
wherein the second acute edge is between the second and third walls.

9. The device of claim 1, wherein the chamber comprises a channel running between the inlet and the outlet provided at first and second ends respectively and comprising first and second channel walls.

10. The device of claim 1, wherein the chamber is disk-shaped having a periphery and a centre and wherein the inlet is annular and arranged around the periphery of the chamber and the outlet is arranged at the centre.

11. The device of claim 1, wherein the chamber has a height which is between 50 to 300 μm.

12. The device of claim 1, wherein the discrete barriers have a width of between 10 and 500 μm.

13. The device of claim 1, wherein the discrete barriers have a length of between 10 and 1,000 μm.

14. The device of claim 1, wherein the discrete barriers are separated from neighbours in a first direction by a first gap of between 10 to 500 μm.

15. The device of claim 1, wherein the discrete barriers are separated from neighbours in a second different direction by a second gap of between 10 to 500 μm.

16. The device of claim 1, wherein the discrete barriers project into the chamber from a floor or a ceiling.

17. The device of claim 1, wherein the discrete barriers have the same shape and/or the same dimensions.

18. The device of claim 1, wherein the discrete barriers are arranged in a periodic array.

19. The device of claim 18, wherein the array is a rectangular array.

20. The device of claim 18, wherein the array is a hexagonal array.

21. An intrauterine insemination kit comprising a motile cell sorting device, the motile cell sorting device comprising:
a chamber;
an inlet and an outlet in fluid communication with the chamber; and
a plurality of discrete barriers disposed in the chamber, wherein each discrete barrier comprises at least one wall and at least one acute edge orientated towards the outlet.

22. A method of using a motile cell sorting device, the motile cell sorting device comprising:
a chamber;
an inlet and an outlet in fluid communication with the chamber; and
a plurality of discrete barriers disposed in the chamber, wherein each discrete barrier comprises at least one wall and at least one acute edge orientated towards the outlet, the method comprising:
supplying a sample comprising motile cells to the inlet;
waiting for a period of time of at least 1 minute; and
after waiting for the period of time, collecting a refined sample from the outlet.

23. The method of claim 22, wherein the period of time is at least 5 minutes.

24. The method of claim 22, wherein the period of time is between 10 to 60 minutes.

25. The method of claim 22, further comprising:
causing the device to be heated to a temperature for incubation.

26. The method of claim 25, wherein the temperature is 37° C.
